(19) United States
(12) Patent Application Publication
(10) Pub. No.: US 2007/0104750 A1
(43) Pub. Date: May 10, 2007

(54) Pesticidally active compositions having enhanced activity

(75) Inventors: Stephen L. Wilson, Indianapolis, IN (US); James E. Dripps, Carmel, IN (US); Raymond E. Boucher JR., Lebanon, IN (US); James M. Gifford, Lebanon, IN (US); Margaret S. Kempe, Greenfield, IN (US); James D. Thomas, Fishers, IN (US); Lei Liu, Carmel, IN (US); Norman R. Pearson, Carmel, IN (US)

Correspondence Address: DOW AGROSCIENCES LLC, 9330 ZIONSVILLE RD, INDIANAPOLIS, IN, 46268, US

(73) Assignee: Dow AgroSciences LLC, Indianapolis, IN (US)

(21) Appl. No.: 11/591,070

(22) Filed: Nov. 1, 2006

Related U.S. Application Data
(60) Provisional application No. 60/732,317, filed on Nov. 1, 2005.

(51) Int. Cl. A01N 25/00; A61K 36/00; A01N 63/02

(52) U.S. Cl. 424/405; 424/725; 424/780; 424/93.46

(57) ABSTRACT

The present invention relates to a pesticidally active composition comprising: 1) at least one pesticidally active ingredient having at least one functionality which is reactive with singlet oxygen, and 2) at least one compound which acts as an activity enhancer, which is distinct from the pesticidally active ingredient.

This application claims the benefit of U.S. Provisional Application No. 60/732,317, filed on Nov. 1, 2005. The present invention relates to pesticidally active compositions having resistance to oxidation and degradation, thus displaying enhanced activity when applied to control pests.

### BACKGROUND

The design of environmentally friendly pesticides has led to products with limited duration of efficacy in the field. This is particularly true with naturally derived pesticides or biopesticides. The short residual activity of these pesticides due to sunlight induced degradation decreases the usefulness of these products and requires an increased amount of pesticide to achieve the desired effect.

Photo-degradation and photo-oxidation of pesticidally active ingredients is well known and methods of protecting active ingredients from such processes have been attempted in various manners, primarily by the use of sunscreens to shield the active ingredient from damaging sunlight, encapsulation of the active ingredient as a barrier to oxygen, or a combination of these two techniques. For example, U.S. Pat. No. 5,246,936 discloses the use of UV-absorbing fluorescent whitening agents to enhance the activity of pesticides, encapsulation with lignin sulfonates is disclosed in U.S. Pat. Nos. 5,939,089; 5,750,467; 5,750,126; 5,529,772; 4,244,728; 4,184,866; and 3,929,453, and interfacial condensation reactions to encapsulate pesticides are disclosed in U.S. Pat. Nos. 4,056,610; 4,497,793 and 4,557,755. Additionally, zein protein has been used to encapsulate a naturally occurring insecticide as disclosed in Journal Agric. Food Chem. “Photostability of Abamectin/Zein Microspheres,” 1997, 45, pgs. 260-262. The use of specific photo-stabilizers to protect agricultural insecticides by the direct photochemical interaction of the photo-stabilizer with the insecticide has also been disclosed in U.S. Pat. No. 4,622,315.

However, the indirect photo-degradation of susceptible pesticidally active ingredients by the action of singlet oxygen has not been adequately addressed by the prior art. Since the absorption of sunlight by the pesticidally active ingredient is not necessary for the occurrence of this type of photo-degradation, sunscreens are not necessarily effective. Therefore, there remains a need to produce other, alternative pesticidally active compositions having improved resistance to degradation and enhanced activity, without the need for encapsulation.

### SUMMARY

The present invention relates to a pesticidally active composition comprising:
1) at least one pesticidally active ingredient having at least one functionality which is reactive with singlet oxygen, and
2) at least one compound which acts as an activity enhancer, which is distinct from the pesticidally active ingredient.

This composition extends the activity or half-life of the pesticidally active ingredient, therefore the same activity can be achieved with a lower amount of the pesticidally active ingredient when compared to a composition in the absence of an activity enhancer.

### BRIEF DESCRIPTION OF DRAWING

FIG. 1 refers to the concentration of DE-175J as a function of irradiation time fit to an exponential decay curve as, described in the Examples.

### DETAILED DESCRIPTION

One aspect of the present invention is a pesticidally active composition comprising at least one pesticidally active ingredient having a functionality which is reactive with singlet oxygen, and at least one compound which acts as an activity enhancer.

A pesticidally active ingredient is herein defined as any compound which shows some pesticidal or biocidal activity, or otherwise participates in the control or limitation of pest populations. Such compounds include fungicides, insecticides, nematocides, miticides, termiticides, rodenticides, molluscides, arthropodicides, herbicides, biocides, as well as pheromones and attractants and the like. Examples of such pesticidally active ingredients can be found in The Pesticide Manual, 12^{th} Edition. The pesticidally active ingredients which can be used in the composition of the present invention are also compounds which are reactive with singlet oxygen. Such compounds include, but are not limited to certain olefins, aromatics, phenols, naphthols, furans, pyrans and other heterocycles containing oxygen; pyrroles, oxazoles, imidazoles, indoles and other heterocycles containing nitrogen; aliphatic, alicyclic and aromatic amines; amino acids, peptides and proteins; and sulfur containing compounds such as mercaptans and sulfides; and the like.

Determination of whether a pesticidally active ingredient is reactive with singlet oxygen can be determined from a simple singlet oxygen reactivity test, comprising irradiating a solution of a pesticidally active ingredient in the presence of a photosensitizer, e.g. methylene blue or rose bengal, with a suitable light source, e.g. a sodium lamp, whose spectral output is capable of allowing light absorption by the photo-sensitizer while bypassing the absorption band of the pesticidally active ingredient. The light source, in combination with the photosensitizer and molecular oxygen, will generate singlet oxygen. The observed degradation of the pesticidally active ingredient by a suitable analytical technique, such as HPLC, is indicative of its reaction with singlet oxygen. This test can be conducted in solvents in which singlet oxygen has a relatively long lifetime, e.g. perdeuterated versions of common solvents and carbon disulfide in order to speed the rate of reaction and thereby maximize the detectability of the degradation. Decay constants for singlet oxygen in various solvents are well known in the art and can be found in J. Phys. Chem. Ref. Data 24: 663-1021 (1995), “Decay Constants for the Decay and Reactions of the Lowest Electronically Excited Singlet State of Molecular Oxygen in Solution. An Expanded and Revised Compilation” by Francis Wilkinson, W. Phillip Helman and Alberta B. Ross; Table 1, entitled Decay Constants for Singlet Oxygen in Various Solvents.

Confirmation of the degradation of the pesticidally active ingredient by singlet oxygen can be obtained further by adding a molecule which is capable of deactivating singlet oxygen by physical or chemical means, i.e. a singlet oxygen quencher, to the pesticidally active ingredient-photosensitizer solution. Suitable singlet oxygen quenchers include, for example DABCO (1,4-diazabicyclo[2.2.2]octane) and beta-carotene. A reduction in the rate of degradation of the pesticidally active ingredient in the presence of a singlet oxygen quencher confirms that the initial degradation was due to the reaction with singlet oxygen.

Exemplary pesticidally active ingredients for use in the composition of the present invention include, but are not limited to, ‘natural products’ which are microorganisms, microbial products, and materials derived or extracted from plants, animals, or mineral-bearing rocks. Natural products include products derived from naturally derived soil dwelling organisms such as actinomycete bacteria, e.g. avermectins and derivatives thereof such as emamectin; and spinosyns and derivatives thereof as disclosed in U.S. Pat. Nos. 5,227,295; 5,670,364; 5,591,606; 6,001,981; 6,143,526; 6,455,504; 6,585,990; 6,919,464; 5,362,634; 5,539,089; and 5,202,242, all of which are herein incorporated by reference. Other natural products include sabadilla or veratrine, pyrethrum or pyrethrin, neem oil or azadirachtin, rotenone, ryania or ryanodine, Bacillus thuringiensis (B.t.), Bacillus subtilis, pheromones, natural attractants and the like. Also included are synthetically produced pesticidally active ingredients which are reactive toward singlet oxygen. Examples include, but are not limited to indoxacarb, imazalil and fenpropimorph.

It is understood by those skilled in the art that any combination of pesticidally active ingredients may also be used in the composition of the present invention as long as the benefits of the composition of the present invention are obtained. In other words, the composition of the present invention can comprise more than one pesticidally active ingredient having at least one functionality reactive with singlet oxygen, or may comprise at least one pesticidally active ingredient having at least one functionality which is reactive with singlet oxygen and at least one other pesticidally active ingredient which does not contain such functionality.

The amount of pesticidally active ingredient within the composition of the present invention will vary depending upon the actual pesticidally active ingredient, the application of the pesticidally active ingredient, the relative amounts of active ingredient and activity enhancer, and the appropriate application levels which are well known to those skilled in the art. In general, the pesticidally active ingredient is present in the composition of the present invention in an amount of from 0.1, generally from 1, preferably from 5, more preferably from 10, even more preferably from 15 and most preferably from 20 to 99, generally to 90, preferably to 85, more preferably to 80 and most preferably to 75 weight percent based on the total weight of the composition of the pesticidally active ingredient, the activity enhancer, and solvent. This composition is then typically further diluted in a formulation which is ready for application.

The composition of the present invention also comprises a compound which acts as an activity enhancer. An activity enhancer is any compound which will enhance the activity of the pesticidally active ingredient, including increasing the half-life of the pesticidally active ingredient, or enabling a lower concentration of the pesticidally active ingredient to achieve the same or similar results as compositions of the pesticidally active ingredient alone. It is thought that the activity enhancer performs this function by deactivating singlet oxygen by physical or chemical means, thereby slowing the degradation of the pesticidally active ingredient. A material falls within the definition of an activity enhancer according to the composition of the present invention if the rate of degradation of the pesticidally active ingredient in the Singlet Oxygen Reactivity Test is slowed in the presence of the material relative to the rate of degradation of the pesticidally active ingredient in the absence of the material.

Generally, the activity enhancer will increase the half life of the pesticidally active ingredient or alternatively, will enable the pesticidally active ingredient to achieve the same pesticidal protection or control at an application level which is less than the concentration required for the same pesticidal protections or control of the pesticidally active ingredient in the absence of the activity enhancer. Specific examples of such activity enhancers include, but are not limited to, carotenoids such as beta-carotene and astaxanthin, chromanols such as alpha-tocopherols (vitamin E) and 6-hydroxy-2,5,7,8-tetramethylchroman-2-carboxylic acid (Trolox™), ascorbates such as ascorbic acid 6-palmitate, amine-containing compounds such as diazabicyclooctane (DABCO), hexamethylenetetramine (HMTA), tetraethylenepentamine (TEPA), and pentaethylenehexamine (PEHA), singlet oxygen-reactive amino acids such as cysteine, methionine, trytophan, histidine, and tyrosine, polypeptides and singlet oxygen deactivating proteins which are relatively rich in singlet oxygen-reactive amino acids, e.g. egg albumin, bovine serum albumin (BSA), and the like. Further examples of activity enhancers can be found in J. Phys. Chem. Ref. Data 24: 663-1021 (1995), “Rate Constants for the Decay and Reactions of the Lowest Electronically Excited Singlet State of Molecular Oxygen in Solution. An Expanded and Revised Compilation” by Francis Wilkinson, W. Phillip Helman and Alberta B. Ross. A material is considered to be an activity enhancer if it slows the rate of degradation of the pesticidally active ingredient as described in the above singlet oxygen reactivity test at a ratio of activity enhancer:active ingredient of less than 20:1, preferably less than 10:1 and most preferably less than 5:1.

The activity enhancer is typically present within the composition of the present invention in an activity enhancing amount. An ‘activity enhancing amount’ is an amount which increases the half life of the pesticidally active ingredient, or alternatively will enable the pesticidally active ingredient to achieve the same control of pests at a level which is less than the amount required for the same pesticidal protection or control of the pesticidally active ingredient in the absence of the activity enhancer. In other words, the activity enhancer will either reduce the rate required for protection or extend the residuality of the pesticidally active ingredient.

Typically, the composition of the present invention comprises a pesticidally active ingredient, activity enhancer, and diluent, such as water, such that the composition is ready for application. Typically the activity enhancer and the pesticidally active ingredient are present within the composition of the present invention at a ratio of activity enhancer to pesticidally active ingredient of from 100:1, generally from 80:1, preferably from 60:1, more preferably from 40:1, even more preferably from 20:1, and most preferably from 10:1 or less.

In one specific embodiment, the present invention relates to a pesticidally active composition comprising:
1) at least one pesticidally active ingredient having at least one functionality which is reactive with singlet oxygen, and
2) at least one compound which acts as an activity enhancer, which is distinct from the pesticidally active ingredient;
   such that the pesticidally active composition provides enhanced pesticidal protection by providing increased half life of the pesticidally active ingredient when compared to compositions without the activity enhancer, or by enabling the pesticidally active ingredient to achieve the same pesticidal protection in a lower amount when compared to the amount required of the pesticidally active ingredient in the absence of the activity enhancer.

In another specific embodiment, the present invention relates to a pesticidally active composition consisting essentially of:
1) at least one pesticidally active ingredient having at least one functionality which is reactive with singlet oxygen, and
2) at least one compound which acts as an activity enhancer, which is distinct from the pesticidally active ingredient.

Other additives and/or adjuvants can also be present within the composition of the present invention, as long as increased residual life or decreased effective rate of the pesticidally active ingredient is obtained.

The composition of the present invention may additionally contain adjuvant surface-active agents to enhance deposition, wetting and penetration of the pesticidally active ingredient onto the target site, e.g. crop, weed, organism or loci. These adjuvant surface-active agents may optionally be employed as a component of the composition of the present invention, or as a tank mix component; the use of and amount desired being well known by those skilled in the art. Suitable adjuvant surface-active agents include, but are not limited to ethoxylated nonyl phenols, ethoxylated synthetic or natural alcohols, salts of the esters of sulfosuccinic acids, ethoxylated organosilicones, ethoxylated fatty amines and blends of surface-active agents with mineral or vegetable oils.

Preferably, the pesticidally active ingredient and activity enhancer are applied in the form of a formulation with a phytologically acceptable carrier. Concentrated formulations can be dispersed in water, or other liquids, for application, or formulations can be dust-like or granular, which can then be applied without further treatment. The formulations can be prepared according to procedures that are conventional in the agricultural chemical art.

The present invention contemplates all vehicles by which the composition of the present invention can be formulated for delivery and use as a pesticide composition, including solutions, suspensions, emulsions, wettable powders and water dispersible granules, emulsifiable concentrates, granules, dusts, baits, and the like. Typically, formulations are applied following dilution of the concentrated formulation with water as aqueous solutions, suspensions or emulsions, or combinations thereof. Such solutions, suspensions or emulsions are produced from water-soluble, water-suspended or water-suspendable, water-emulsified or water-emulsifiable formulations or combinations thereof which are solids, including and usually known as wettable powders or water dispersible granules; or liquids including and usually known as emulsifiable concentrates, aqueous suspensions or suspension concentrates, and aqueous emulsions or emulsions in water, or mixtures thereof such as suspension-emulsions. As will be readily appreciated, any material to which this composition can be added may be used, provided they yield the desired utility without significant interference with the desired activity of the pesticidally active ingredients as pesticidal agents and improved residual lifetime or decreased effective concentration is achieved.

Wettable powders, which may be compacted to form water dispersible granules, comprise an intimate mixture of one or more of the pesticidally active ingredients, an inert carrier and surfactants. The concentration of the pesticidally active ingredient in the wettable powder is usually from about 10 percent to about 90 percent by weight based on the total weight of the wettable powder, more preferably about 25 weight percent to about 75 weight percent. In the preparation of wettable powder formulations, the pesticidally active ingredients can be compounded with any finely divided solid, such as prophyllite, talc, chalk, gypsum, Fuller's earth, bentonite, attapulgite, starch, casein, gluten, montmorillonite clays, diatomaceous earths, purified silicates or the like. In such operations, the finely divided carrier and surfactants are typically blended with the compound(s) and milled.

Emulsifiable concentrates of the pesticidally active ingredient comprise a convenient concentration, such as from about 10 weight percent to about 50 weight percent of the pesticidally active ingredient, in a suitable liquid, based on the total weight of the concentrate. The pesticidally active ingredients are dissolved in an inert carrier, which is either a water miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used, such as, for example, terpenic solvents, including rosin derivatives, aliphatic ketones, such as cyclohexanone, and complex alcohols, such as 2-ethoxyethanol.

Emulsifiers which can be advantageously employed herein can be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters esterified with the polyol or polyoxyalkylene. Cationic emulsifiers include quaternary ammonium compounds and fatty amine salts. Anionic emulsifiers include the oil-soluble salts (e.g., calcium) of alkylaryl sulfonic acids, oil-soluble salts of sulfated polyglycol ethers and appropriate salts of phosphated polyglycol ether.

Representative organic liquids which can be employed in preparing emulsifiable concentrates are the aromatic liquids such as xylene, propyl benzene fractions; or mixed naphthalene fractions, mineral oils, substituted aromatic organic liquids such as dioctyl phthalate; kerosene; dialkyl amides of various fatty acids, particularly the dimethyl amides; and glycol ethers such as the n-butyl ether, ethyl ether or methyl ether of diethylene glycol, and the methyl ether of triethylene glycol and the like. Mixtures of two or more organic liquids may also be employed in the preparation of the emulsifiable concentrate. Surface-active emulsifying agents are typically employed in liquid formulations and in an amount of from 0.1 to 20 percent by weight based on the combined weight of the emulsifying agents. The formulations can also contain other compatible additives, for example, plant growth regulators and other biologically active compounds used in agriculture.

Aqueous suspensions comprise suspensions of one or more water-insoluble pesticidally active ingredients dispersed in an aqueous vehicle at a concentration in the range from about 5 to about 50 weight percent, based on the total weight of the aqueous suspension. Suspensions are prepared by finely grinding one or more of the pesticidally active ingredients, and vigorously mixing the ground material into a vehicle comprised of water and surfactants chosen from the same types discussed above. Other components, such as inorganic salts and synthetic or natural gums, may also be added to increase the density and viscosity of the aqueous vehicle. It is often most effective to grind and mix at the same time by preparing the aqueous mixture and homogenizing it in an implement such as a sand mill, ball mill, or piston-type homogenizer.

Aqueous emulsions comprise emulsions of one or more water-insoluble pesticidally active ingredients emulsified in an aqueous vehicle at a concentration typically in the range from about 5 to about 50 weight percent, based on the total weight of the aqueous emulsion. If the pesticidally active ingredient is a solid it must be dissolved in a suitable water-immiscible solvent prior to the preparation of the aqueous emulsion. Emulsions are prepared by emulsifying the liquid pesticidally active ingredient or water-immiscible solution thereof into an aqueous medium typically with inclusion of surfactants that aid in the formation and stabilization of the emulsion as described above. This is often accomplished with the aid of vigorous mixing provided by high shear mixers or homogenizers.

The compositions of the present invention can also be granular formulations, which are particularly useful for applications to the soil. Granular formulations usually contain from about 0.5 to about 10 weight percent, based on the total weight of the granular formulation of the pesticidally active ingredient(s), dispersed in an inert carrier which consists entirely or in large part of coarsely divided inert material such as attapulgite, bentonite, diatomite, clay or a similar inexpensive substance. Such formulations are usually prepared by dissolving the pesticidally active ingredients in a suitable solvent and applying it to a granular carrier which has been preformed to the appropriate particle size, in the range of from about 0.5 to about 3 mm. A suitable solvent is a solvent in which the compound is substantially or completely soluble. Such formulations may also be prepared by making a dough or paste of the carrier and the compound and solvent, and crushing and drying to obtain the desired granular particle.

Dusts can be prepared by intimately mixing one or more of the pesticidally active ingredients in powdered form with a suitable dusty agricultural carrier, such as, for example, kaolin clay, ground volcanic rock, and the like. Dusts can suitably contain from about 1 to about 10 weight percent of the compounds, based on the total weight of the dust.

The formulations may additionally contain adjuvant surfactants to enhance deposition, wetting and penetration of the pesticidally active ingredients onto the target site such as a crop or organism. These adjuvant surfactants may optionally be employed as a component of the formulation or as a tank mix. The amount of adjuvant surfactant will typically vary from 0.01 to 1.0 percent by volume, based on a spray-volume of water, preferably 0.05 to 0.5 volume percent. Suitable adjuvant surfactants include, but are not limited to ethoxylated nonyl phenols, ethoxylated synthetic or natural alcohols, salts of the esters of sulfosuccinic acids, ethoxylated organosilicones, ethoxylated fatty amines and blends of surfactants with mineral or vegetable oils.

The activity enhancer can be added at any time prior to application of the pesticidally active ingredient or applied individually as a distinct application. Preferably the activity enhancer is incorporated into the formulation as desired or added later as a tank mix component.

The formulations can also include any combination of pesticidal compounds. Such additional pesticidal compounds may be other fungicides, insecticides, nematocides, miticides, arthropodicides, molluscicides, bactericides, attractants, pheromones or combinations thereof that are compatible with the compositions of the present invention in the medium selected for application, and are not antagonistic to the enhanced activity of the desired pesticidally active ingredients. Accordingly, in such embodiments, the other pesticidal compound is employed as a supplemental toxicant for the same or for a different pesticidal use. The desired pesticidally active ingredients, and the pesticidal compound in the combination can generally be present in a weight ratio of from 1:100 to 100:1.

One specific embodiment of the present invention is a composition comprising:
1) at least one pesticidally active ingredient having at least one functionality which is reactive with singlet oxygen,
2) at least one compound which acts as an activity enhancer which is distinct from the pesticidally active ingredient, and an agriculturally acceptable carrier.

Another specific embodiment of the present invention is a composition comprising:
1) at least one pesticidally active ingredient having at least one functionality which is reactive with singlet oxygen,
2) at least one compound which acts as an activity enhancer which is distinct from the pesticidally active ingredient, and
3) an agriculturally acceptable carrier,
wherein the pesticidally active component is not encapsulated.

Yet another specific embodiment of the present invention is a composition consisting essentially of:
1) at least one pesticidally active ingredient having at least one functionality which is reactive with singlet oxygen,
2) at least one compound which acts as an activity enhancer which is distinct from the pesticidally active ingredient, and
3) an agriculturally acceptable carrier.

Another embodiment of the present invention is the use of the composition of the present invention in pesticidal applications to control, prevent or eliminate unwanted living organisms, e.g. fungi, bacteria or other microbes, weeds, insects, rodents and other pests. This would include its use for protection of a plant against attack by a phytopathogenic organism or the treatment of a plant already infested by a phytopathogenic organism, comprising applying the composition of the present invention, to soil, a plant, a part of a plant, foliage, flowers, fruit, and/or seeds in a disease inhibiting and phytologically acceptable amount. The term “disease inhibiting and phytologically acceptable amount” refers to an amount of a pesticidally active ingredient that kills or inhibits the plant disease for which control is desired, but is not significantly toxic to the plant. This amount will generally be from about 0.1 to about 1000 ppm (parts per million), with 1 to 500 ppm being preferred. The exact concentration of pesticidally active ingredient required varies with the fungal disease to be controlled, the type of formulation employed, the method of application, the particular plant species, climate conditions, and the like, as is well known in the art. A suitable application rate is typically in the range from about 0.1 to about 4 pounds/acre (about 0.01 to 0.45 grams per square meter, g/m²).

Additionally, the composition of the present invention is useful for the control of insects or other pests, e.g. rodents. Therefore, the present invention also is directed to a method for inhibiting an insect or pest which comprises applying the composition of the present invention to a locus of the insect or pest in an insect-inhibiting amount. The “locus” of insects or pests is a term used herein to refer to the environment in which the insects or pests live or where their eggs are present, including the air surrounding them, the food they eat, or objects which they contact. For example, insects which eat or contact edible or ornamental plants can be controlled by applying the active compound to plant parts such as the seed, seedling, or cutting which is planted, the leaves, stems, fruits, grain, or roots, or to the soil in which the roots are growing. It is contemplated that the pesticidally active ingredients and compositions containing such, might also be useful to protect textiles, paper, stored grain, seeds, domesticated animals, buildings or human beings by applying an active compound to or near such objects. The term “inhibiting an insect or pest” refers to a decrease in the numbers of living insects or pests, or a decrease in the number of viable insect eggs, including methods of mating disruption using attractants and/or pheromones. The extent of reduction accomplished by a pesticidally active ingredient depends, upon the application rate of the pesticidally active ingredient, the particular pesticidally active ingredient used, and the target insect or pest species. At least an inactivating amount should be used. The terms “insect or pest-inactivating amount” are used to describe the amount, which is sufficient to cause a measurable reduction in the treated insect or pest population, as is well known in the art. Generally an amount in the range from about 1 to about 1000 ppm pesticidally active ingredient is used.

The locus to which a compound is applied can be any locus inhabited by an insect, mite or pest, for example, vegetable crops, fruit and nut trees, grape vines, ornamental plants, domesticated animals, the interior or exterior surfaces of buildings, and the soil around buildings.

Because of the unique ability of insect eggs to resist toxicant action, repeated applications may be desirable to control newly emerged larvae, as is true of other known insecticides and acaricides.

Additionally, another aspect of the present invention relates to the use of the composition of the present invention as herbicides. The term herbicide is used herein to mean an active ingredient that kills, controls or otherwise adversely modifies the growth of plants. An herbicidally effective or vegetation controlling amount is an amount of active ingredient which causes an adversely modifying effect and includes deviations from natural development, killing, regulation, desiccation, retardation, and the like. The terms plants and vegetation include emerging seedlings and established vegetation.

Herbicidal activity is exhibited when they are applied directly to the locus of the undesirable plant thereof at any stage of growth or before emergence of the weeds. The effect observed depends upon the plant species to be controlled, the stage of growth of the plant, the particle size of solid components, the environmental conditions at the time of use, the specific adjuvants and carriers employed, the soil type, and the like, as well as the amount of chemical applied. These and other factors can be adjusted as is known in the art to promote selective herbicidal action. Generally, it is preferred to apply such herbicides post emergence to relatively immature undesirable vegetation to achieve the maximum control of weeds.

Application rates of about 1 to about 2,000 g/Ha are generally employed in post emergence operations; for preemergence applications, rates of about 1 to about 2,000 g/Ha are generally employed. The higher rates designated generally give non-selective control of a broad variety of undesirable vegetation. The lower rates typically give selective control and can be employed in the locus of crops.

The actual amount of pesticidally active ingredient to be applied to loci of disease, insects and mites, or weeds is well known in the art and can readily be determined by those skilled in the art in view of the teachings above.

The composition of the present invention surprisingly offers increased residual lifetime to the pesticidally active ingredient, thus offering improved preventative measures for disease, insects, mites or weed infestations.

### EXAMPLES

These examples are provided to further illustrate the invention and are not meant to be construed as limiting. As disclosed herein, all temperatures are given in degrees Celsius and all percentages are weight percentages unless otherwise stated.

A. Determination of Pesticidally Active Ingredient Susceptibility to Singlet Oxygen Degradation.

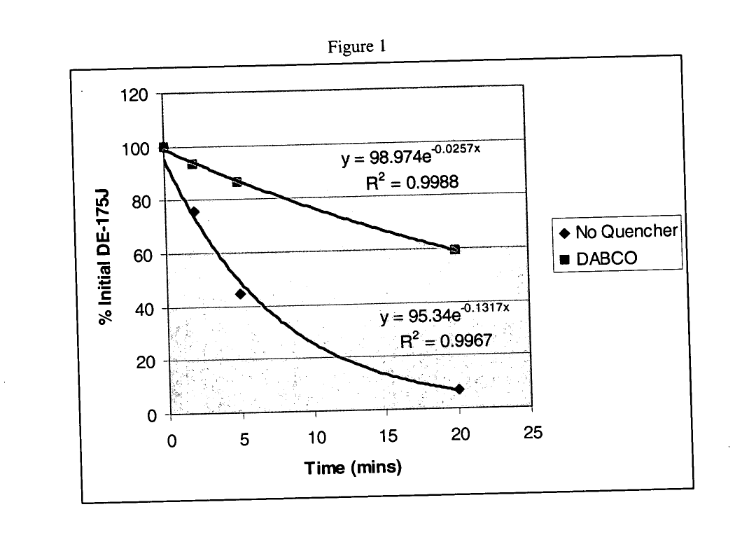

The major component of spinetoram identified herein as DE-175J (5.0×10⁻⁴M) was combined with rose bengal (5.0×10⁻⁵M, bis(triethylammonium)salt, Aldrich) in acetonitrile. The solution was irradiated with a Philips 150 watt high pressure sodium lamp (sample was ca. 20 cm from lamp). The degradation of DE-175J as a result of the irradiation was monitored by HPLC. During the irradiation the singlet oxygen concentration is in a steady state condition (it doesn't change) and the rate equation of loss of DE-175J is, therefore, 1st order. The concentration of DE-175J as a function of irradiation time was fit to an exponential decay curve using Microsoft Excel to calculate the rate constant for the degradation. The result is shown in FIG. 1; a value of 0.1317 min⁻¹ was obtained. The experiment was repeated with the inclusion of the singlet oxygen quencher DABCO (1.0×10⁻³M, diazabicyclooctane Aldrich). The ratio of the rate constant without DABCO to that with it (kwo/kw) was calculated and is shown in Table 1. A value greater than one indicates a slowing of the degradation due to singlet oxygen in the presence of the quencher. A kwo/kw value of 5.1 was found for DE-175J indicating that the degradation is about 5 times or 500% faster without DABCO, thus confirming DE-175J's susceptibility to singlet oxygen degradation. Table 1 also shows the results obtained from analogous experiments for some other pesticidally active ingredients that were found to be degraded by singlet oxygen.

TABLE 1

| | Weight Ratio Pesticidally | | |
|---|---|---|---|
| Pesticidally | Active | | |
| Active Ingredient | Ingredient/DABCO | Solvent | kwo/kw |
| DE-175 J | 3.3:1 | acetonitrile | 5.1 |
| Abamectin | 7.8:1 | acetonitrile-d3 | 37.2 |
| Imazalil | 2.6:1 | acetonitrile | 4.4 |
| Fenpropimorph | 2.7:1 | acetonitrile | 7.7 |

B. Determination of Potential Suitability of an Ingredient as an Activity Enhancer.

The procedure of A. above was repeated with the substitution of TEPA (tetraethylenepentamine) (5.92×10⁻⁴ M, Aldrich) for DABCO. A kwo/kw value of 1.9 was obtained, indicating that DE-175J degrades nearly twice as fast without the TEPA. This result is shown in Table 2. Also included in Table 2 are additional examples including the comparative example of zein (Freeman Industries) showing that even at high levels (20:1 relative to abamectin) this material does not slow the degradation from singlet oxygen.

TABLE 2

| I. Pesticidally | II. Potential | | | |
|---|---|---|---|---|
| Active | Activity | Weight | | |
| Ingredient | Enhancer | Ratio I/II | Solvent | kwo/kw |
| DE-175 J | TEPA | 3.3:1 | acetonitrile | 1.9 |
| DE-175 J | Tryptophan | 1:10 | methanol | 1.3 |
| DE-175 J | Tryptophan | 1:1 | methanol | 1.15 |
| DE-175 J | Zein | 1:5 | isopropanol(85)/ | 1.0 |
| | | | water(15) | |
| Abamectin | Zein | 1:20 | isopropanol(85)/ | 1.0 |
| | | | water(15) | |

Examples

Efficacy experiments were conducted by preparing the appropriate concentration of a) pesticidally active ingredient (control), b) activity enhancer, (control) and c) pesticidally active ingredient+activity enhancer, in water. These solutions, plus a water-only control, were applied to potted pepper plants (Capsicum annuum) using a Mandel track sprayer calibrated to deliver the equivalent of 400 L/Ha of spray. Treated plants were allowed to dry and then were aged outdoors in natural sunlight or under a set of lamps emitting ultraviolet light at levels comparable to natural sunlight. At the appropriate time after treatment, 2.5 cm diameter disks were cut from treated leaves. One leaf disk was laced in each well of a 32 well plastic tray, which also contained a thin layer of agar to provide moisture. There were 8 replicate disks per treatment. Each well was infested with three second instar beet armyworm (Spodoptera exigua) larvae, and the well was sealed with plastic film. Larvae were held in an environmental chamber at 25° C./40 percent relative humidity. At 48 hours after infestation, the larvae were graded for mortality. A larva was considered dead if it could not move after being prodded, and the percent dead were calculated.

Formulation for Spinetoram SC of Examples 6, 7, 9-19, 25, 26 and Controls thereof.

| Ingredient | Weight percent |
|---|---|
| Spinetoram | 10 |
| Antifoam B (available from Dow Corning) | 2 |
| Kelzan (xanthan gum) | 0.07 |
| Lomar PWA (available from Cognis Corp.) | 2 |
| Pluronic P-105 (available from BASF) | 3 |
| Propylene glycol | 4 |
| Proxel GXL (available from Avecia Biocides) | 0.2 |
| Veegum (available from RT Vanderbilt) | 0.42 |
| Water | 78.31 |

Spinetoram is a mixture of (2R,3aR,5aR,5bS,9S,13S,14R,16aS,16bR)-13-{[(2R,5S,6R)-5-(dimethylamino)-6-methyltetrahydro-2H-pyran-2-yl]oxy}-9-ethyl-14-methyl-7,15-dioxo-2,3,3a,4,5,5a,5b,6,7,9,10,11,12,13,14,15,16a,16b-octadecahydro-1H-as-indaceno[3,2-d]oxacyclododecin-2-yl 6-deoxy-3-O-ethyl-2,4-di-O-methyl-α-L-mannopyranoside [DE-175J] and (2S,3aR,5aS,5bS,9S,13S,14R,16aS,16bS)-13-{[(2R,5S,6R)-5-(dimethylamino)-6-methyltetrahydro-2H-pyran-2-yl]oxy}-9-ethyl-4,14-dimethyl-7,15-dioxo-2,3,3a,5a,5b,6,7,9,10,11,12,13,14,15,16a,16b-hexadecahydro-1H-as-indaceno[3,2-d]oxacyclododecin-2-yl 6-deoxy-3-O-ethyl-2,4-di-O-methyl-α-L-mannopyranoside in the proportion 50-90% to 50-10%.

Formulation for Spinetoram with TEPA Oleamide SC (Example 18) Ingredient Weight Percent

| Ingredient | Weight Percent |
|---|---|
| Spinetoram | 10 |
| TEPA Oleamide | 5 |
| Pluronic P-105 (BASF) | 2 |
| Lomar PWA (Cognis Corp) | 2 |
| Antifoam B (Dow Corning) | 2 |
| Water | 79 |

Formulation for Pyrethrum EC (Example 23) and Control thereof.

| Ingredient | wt % |
|---|---|
| Pyrethrum (43.5%) | 10 |
| Aromatic 100 (ExxonMobil) | 21 |
| Polyglycol ™ P2000 (Dow Chemical) | 63 |
| Agrimul ™ Hydro D (Cognis Corp) | 3 |
| Agrimul ™ Lipo D (Cognis Corp) | 3 |

Formulation for beta-Carotene-coated Spinosad SC (Example 24)

The Beta-Carotene-coated Spinosad sample was prepared using a Wuster Dryer process where Spinosad solid particles were kept suspended and dispersed in the chamber by continuous air flow and a beta-carotene solution was introduced by a spray nozzle located in the center of the chamber. The chamber temperature was maintained at 55-60° C. A saturated beta-carotene solution in methylene chloride was first prepared as a coating material. Spinosad technical was air-milled to ca. 3.5 microns, and then loaded into the Wuster dryer. After the chamber temperature and air flow stabilized, the coating solution was slowly fed into the chamber. The feed rate was ˜10 ml/min and the amount of coating material (beta-carotene) was 3% w/w of Spinosad. About 20 minutes of time was allowed for further drying and curing after the completion of coating material injection, before the coated powders were collected. An aqueous suspension concentrate formulation containing 10% of the coated Spinosad was prepared with the composition shown below. The control sample used for comparison was prepared by the identical procedure described above except without introducing the coating materials.

| Ingredient | Weight Percent |
|---|---|
| Spinosad | 10 |
| Beta-Carotene | 0.3 |
| Pluronic P-105 (BASF) | 3 |
| Morwet D-425 (Akzo Nobel) | 2 |
| Antifoam B (Dow Corning) | 1 |
| Water | 83.7 |

Formulation for Spinetoram with Ascorbic Acid 6-Palmitate EC (Example 25)

| Ingredient | Weight Percent |
|---|---|
| Spinetoram | 5 |
| Ascorbic acid 6-palmitate (Aldrich) | 5 |
| Atlox 4913 (Uniqema) | 2.5 |
| N-methyl pyrrolidinone | 87.5 |

For a control, the above Spinetoram EC was prepared minus the ascorbic acid 6-palmitate, with an additional 5% N-methylpyrrolidinone as the balance ingredient.

The results of the Examples are listed in Table 3.

Example 1 demonstrates the effect of 1000 ppm DABCO on improving the efficacy of SUCCESS™ insecticide at 5 and 9 days after treatment.

Examples 2 and 3 demonstrate the effect of 1000 and 500 ppm TEPA on improving the efficacy of SUCCESS™ insecticide at 5, 7, and 9 days after treatment.

Example 4 demonstrates the effect of 1000 ppm DABCO on improving the efficacy of AVAUNT™ insecticide at 5, 7, and 9 days after treatment.

Example 5 demonstrates the effect of 1000 ppm TEPA on improving the efficacy of DENIM™ insecticide at 2, 5, 7, and 9 days after treatment.

Example 6 demonstrates the effect of 1000 ppm HMTA on improving the efficacy of spinetoram insecticide at 9 days after treatment.

Example 7 demonstrates the effect of 1000 ppm TEPA on improving the efficacy of spinetoram insecticide at 9 days after treatment.

Example 8 demonstrates the effect of 1000 ppm TEPA on improving the efficacy of DIPEL™ insecticide at 2 and 5 days after treatment.

Example 9 demonstrates the effect of 1000 ppm PEHA on improving the efficacy of spinetoram insecticide at 3 and 7 days after treatment.

Example 10 demonstrates the effect of 1000 ppm TETA on improving the efficacy of spinetoram insecticide at 3 and 7 days after treatment.

Examples 11 and 12 demonstrate the effect of 1000 and 10,000 ppm bovine serum albumin (as 96% powder) on improving the efficacy of spinetoram insecticide at 4 and 7 days after treatment.

Examples 13 and 14 demonstrate the effect of 1000 and 10,000 ppm bovine serum albumin (as 30% aqueous suspension) on improving the efficacy of spinetoram insecticide at 4 and 7 days after treatment.

Example 15 demonstrates the effect of 1000 ppm TROLOX™ on improving the efficacy of spinetoram insecticide at 4 and 7 days after treatment.

Example 16 demonstrates the effect of 1000 ppm chitosan on improving the efficacy of spinetoram insecticide at 5 days after treatment.

Example 17 demonstrates the effect of 1000 ppm egg albumin on improving the efficacy of spinetoram insecticide at 5 days after treatment.

Example 18 demonstrates the effect of adding TEPA oleamide to a suspension concentrate formulation of spinetoram insecticide; efficacy was improved at 5 days after treatment.

Example 19 demonstrates the effect of 1000 ppm mancozeb (as DITHANE™) on improving the efficacy of spinetoram insecticide at 4 and 7 days after treatment.

Example 20 demonstrates the effect of 1000 ppm bovine serum albumin (as 30% aqueous suspension) on improving the efficacy of DENIM™ insecticide at 4 and 7 days after treatment.

Example 21 demonstrates the effect of 1000 ppm bovine serum albumin (as 30% aqueous suspension) on improving the efficacy of DIPEL™ insecticide at 2 days after treatment.

Example 22 demonstrates the effect of 1000 ppm bovine serum albumin (as 96% powder) on improving the efficacy of DIPEL™ insecticide at 2 and 7 days after treatment.

Example 23 demonstrates the effect of 1000 ppm TEPA on improving the efficacy of pyrethrum insecticide in an emulsifiable concentrate formulation at 2 and 3 days after treatment.

Example 24 demonstrates the effect of adding beta-carotene to a suspension concentrate formulation of spinosad insecticide; efficacy was improved at 4 and 7 days after treatment.

Example 25 demonstrates the effect of adding ascorbic acid 6-palmitate to an emulsifiable concentrate formulation of spinosad insecticide, efficacy was improved at 6 days after treatment.

Example 26 demonstrates the effect of 1000 ppm TEPA on the efficacy of spinetoram insecticide. Addition of TEPA enabled 63 ppm of spinetoram to achieve greater mortality at 7 and 9 days after treatment than 94 ppm or 125 ppm of spinetoram without TEPA.

TABLE 3

| | | Percent mortality | | | | | | | |
|---|---|---|---|---|---|---|---|---|---|
| | | 2 | 3 | 4 | 5 | 6 | 7 | 8 | 9 |
| Example Number | Treatment | DAT | DAT | DAT | DAT | DAT | DAT | DAT | DAT |
| Pesticidally active | SUCCESS ™, 250 ppm ai | — | — | — | 21 | — | 17 | — | 8 |
| ingredient Control for | | | | | | | | | |
| Examples 1, 2, & 3 | | | | | | | | | |
| Example 1 | SUCCESS ™, 250 ppm ai + DABCO, 1000 ppm | — | — | — | 63 | — | 17 | — | 13 |
| Example 2 | SUCCESS ™, 250 ppm ai + TEPA, 1000 ppm | — | — | — | 83 | — | 58 | — | 50 |
| Example 3 | SUCCESS ™, 250 ppm ai + TEPA, 500 ppm | — | — | — | 83 | — | 42 | — | 38 |
| Activity enhancer | DABCO, 1000 ppm | — | — | — | 4 | — | 0 | — | 0 |
| Controls for | TEPA, 1000 ppm | — | — | — | 0 | — | 0 | — | 0 |
| Examples 1, 2, & 3 | | | | | | | | | |
| Pesticidally active | AVAUNT ™, 125 ppm ai | 100 | — | — | 83 | — | 8 | — | 21 |
| ingredient Control for | | | | | | | | | |
| Example 4 | | | | | | | | | |
| Example 4 | AVAUNT ™, 125 ppm ai + DABCO, 1000 ppm | 100 | — | — | 88 | — | 58 | — | 38 |
| Activity enhancer | DABCO, 1000 ppm | 0 | — | — | 0 | — | 0 | — | 0 |
| Control for Example 4 | | | | | | | | | |
| Pesticidally active | DENIM ™, 25 ppm ai | 79 | — | — | 42 | — | 17 | — | 13 |
| ingredient Control for | | | | | | | | | |
| Example 5 | | | | | | | | | |
| Example 5 | DENIM ™, 25 ppm ai + TEPA, 1000 ppm | 100 | — | — | 100 | — | 92 | — | 100 |
| Activity enhancer | TEPA, 1000 ppm | 0 | — | — | 0 | — | 0 | — | 0 |
| Control for Example 5 | | | | | | | | | |
| Pesticidally active | Spinetoram SC, 125 ppm ai | — | — | — | — | — | — | — | 58 |
| ingredient Control for | | | | | | | | | |
| Examples 6 & 7 | | | | | | | | | |
| Example 6 | Spinetoram SC, 125 ppm ai + HMTA, 1000 ppm | — | — | — | — | — | — | — | 71 |
| Example 7 | Spinetoram SC, 125 ppm ai + TEPA, 1000 ppm | — | — | — | — | — | — | — | 92 |
| Activity enhancer | HMTA, 1000 ppm | — | — | — | — | — | — | — | 4 |
| Controls for | TEPA, 1000 ppm | — | — | — | — | — | — | — | 0 |
| Examples 6 & 7 | | | | | | | | | |
| Pesticidally active | DIPEL ™, 11.2 g product/L | 0 | — | — | 0 | — | — | — | — |
| ingredient Control for | | | | | | | | | |
| Example 8 | | | | | | | | | |
| Example 8 | DIPEL ™, 11.2 g product/L + TEPA, 1000 ppm | 83 | — | — | 58 | — | — | — | — |
| Activity enhancer | TEPA, 1000 ppm | 0 | — | — | 0 | — | — | — | — |
| Control for Example 8 | | | | | | | | | |
| Pesticidally active | Spinetoram SC, 125 ppm ai | — | 96 | — | — | — | 38 | — | — |
| ingredient Control for | | | | | | | | | |
| Examples 9 & 10 | | | | | | | | | |
| Example 9 | Spinetoram SC, 125 ppm ai + PEHA, 1000 ppm | — | 100 | — | — | — | 96 | — | — |
| Example 10 | Spinetoram SC, 125 ppm ai + TETA, 1000 ppm | — | 100 | — | — | — | 71 | — | — |
| Activity enhancer | PEHA, 1000 ppm | — | 4 | — | — | — | 0 | — | — |
| Control for Examples | TETA, 1000 ppm | — | 4 | — | — | — | 0 | — | — |
| 9 & 10 | | | | | | | | | |
| Pesticidally active | Spinetoram SC, 125 ppm ai | — | — | 63 | — | — | 54 | — | — |
| ingredient Control for | | | | | | | | | |
| Examples 11, 12, 13, | | | | | | | | | |
| 14, & 15 | | | | | | | | | |
| Example 11 | Spinetoram SC, 125 ppm ai + BSA (as 96% powder), | — | — | 79 | — | — | 63 | — | — |
| | 1000 ppm | | | | | | | | |
| Example 12 | Spinetoram SC, 125 ppm ai + BSA (as 96% powder), | — | — | 88 | — | — | 71 | — | — |
| | 10,000 ppm | | | | | | | | |
| Example 13 | Spinetoram SC, 125 ppm ai + BSA (as 30% aqueous | — | — | 83 | — | — | 71 | — | — |
| | suspension), 1000 ppm | | | | | | | | |
| Example 14 | Spinetoram SC, 125 ppm ai + BSA (as 30% aqueous | — | — | 92 | — | — | 83 | — | — |
| | suspension), 10,000 ppm | | | | | | | | |
| Example 15 | Spinetoram SC, 125 ppm ai + Trolox ™, 1000 ppm | — | — | 71 | — | — | 58 | — | — |
| Activity enhancer | BSA (as 96% powder), 10,000 ppm | — | — | 0 | — | — | 0 | — | — |
| Controls for | BSA (as 30% aqueous suspension), 10,000 ppm | — | — | 0 | — | — | 0 | — | — |
| Examples 11, 12, 13, | Trolox ™, 1000 ppm | — | — | 0 | — | — | 0 | — | — |
| 14, & 15 | | | | | | | | | |
| Pesticidally active | Spinetoram SC, 125 ppm ai | — | 92 | — | 25 | — | — | 25 | — |
| ingredient Control for | | | | | | | | | |
| Examples 16, 17, & | | | | | | | | | |
| 18 | | | | | | | | | |
| Example 16 | Spinetoram SC, 125 ppm ai + Chitosan, 1000 ppm | — | 92 | — | 33 | — | — | 13 | — |
| Example 17 | Spinetoram SC, 125 ppm ai + Egg Albumin, 1000 ppm | — | 92 | — | 88 | — | — | 25 | — |
| Example 18 | Spinetoram with TEPA oleamide SC, 125 ppm ai | — | 88 | — | 42 | — | — | 8 | — |
| Activity enhancer | Chitosan, 1000 ppm | — | 0 | — | 0 | — | — | 0 | — |
| Controls for | Egg albumin, 1000 ppm | — | 0 | — | 0 | — | — | 0 | — |
| Examples 16, 17, & | | | | | | | | | |
| 18 | | | | | | | | | |
| Pesticidally active | Spinetoram SC, 125 ppm ai | — | — | 21 | — | — | 21 | — | — |
| ingredient Control for | | | | | | | | | |
| Example 19 | | | | | | | | | |
| Example 19 | Spinetoram SC, 125 ppm ai + DITHANE ™, 1000 ppm | — | — | 33 | — | — | 42 | — | — |
| | ai | | | | | | | | |
| Activity enhancer | DITHANE ™, 1000 ppm ai | — | — | 0 | — | — | — | — | — |
| Control for Example | | | | | | | | | |
| 19 | | | | | | | | | |
| Pesticidally active | DENIM ™, 25 ppm ai | — | — | 50 | — | — | 27 | — | — |
| ingredient Control | | | | | | | | | |
| for Example 20 | | | | | | | | | |
| Example 20 | DENIM ™, 25 ppm ai + BSA (as 30% aqueous | — | — | 79 | — | — | 64 | — | — |
| | suspension), 1000 ppm | | | | | | | | |
| Pesticidally active | DIPEL ™, 11.2 g product/L | 38 | — | — | — | — | 33 | — | — |
| ingredient Control | | | | | | | | | |
| for Examples 21 & | | | | | | | | | |
| 22 | | | | | | | | | |
| Example 21 | DIPEL ™, 11.2 g product/L + BSA (as 30% aqueous | 67 | — | — | — | — | 21 | — | — |
| | suspension), 1000 ppm? | | | | | | | | |
| Example 22 | DIPEL ™, 11.2 g product/L + BSA (as 96% powder), | 71 | — | — | — | — | 58 | — | — |
| | 1000 ppm? | | | | | | | | |
| Pesticidally active | Pyrethrum EC, 1000 ppm ai | 0 | 0 | — | — | — | — | — | — |
| ingredient Control for | | | | | | | | | |
| Example 23 | | | | | | | | | |
| Example 23 | Pyrethrum EC, 1000 ppm ai + TEPA, 1000 ppm | 38 | 29 | — | — | — | — | — | — |
| Pesticidally active | Spinosad SC, 250 ppm ai | — | — | 79 | — | — | 8 | — | — |
| ingredient Control for | | | | | | | | | |
| Example 24 | | | | | | | | | |
| Example 24 | Spinosad with Beta-carotene coating SC, 250 ppm ai | — | — | 92 | — | — | 25 | — | — |
| Pesticidally active | Spinetoram EC, 125 ppm ai | — | — | 83 | — | 0 | — | — | — |
| ingredient Control for | | | | | | | | | |
| Example 25 | | | | | | | | | |
| Example 25 | Spinetoram with Ascorbic palmitate EC, 125 ppm ai | — | — | 79 | — | 42 | — | — | — |
| Pesticidally active | Spinetoram SC, 125 ppm ai | 100 | — | 100 | — | — | 38 | — | 25 |
| ingredient Controls | Spinetoram SC, 94 ppm ai | 100 | — | 92 | — | — | 13 | — | 13 |
| and concentrations | Spinetoram SC, 63 ppm ai | 92 | — | 83 | — | — | 8 | — | 0 |
| for Example 26 | | | | | | | | | |
| Example 26 | Spinetoram SC, 125 ppm ai + TEPA, 1000 ppm | 100 | — | 100 | — | — | 83 | — | 79 |
| | Spinetoram SC, 94 ppm ai + TEPA, 1000 ppm | 100 | — | 100 | — | — | 79 | — | 63 |
| | Spinetoram SC, 63 ppm ai + TEPA, 1000 ppm | 100 | — | 92 | — | — | 79 | — | 29 |

DAT = days after treatment
ai = active ingredient
ppm = parts per million
SC = suspension concentrate formulation
EC = emulsifiable concentrate formulation
“—” = not tested
BSA = Bovine Serum Albumin available from Sigma
Chitosan available from Aldrich
HMTA = hexamethylenetetramine available from Aldrich
TEPA = tetraethylenepentamine available from Aldrich
PEHA = pentaethylenehexamine available from Aldrich
TROLOX ™ = 6-hydroxy-2,5,7,8-tetramethylchroman-2-carboxylic acid available from Aldrich
SUCCESS ™ contains spinosad insecticide and is available from Dow AgroSciences
DENIM ™- contains emamectin benzoate insecticide and is available from Syngenta
AVAUNT ™ contains indoxacarb insecticide and is available from DuPont
DIPEL ™ contains Bacillus thuringiensis kurstaki endotoxin insecticide available from Valent Biosciences
DITHANE ™ contains mancozeb fungicide available from Dow AgroSciences
Egg Albumin available from Sigma
DABCO = diazabicyclooctane available from Aldrich
TEPA Oleamide = prepared from the reaction of methyl oleate with TEPA as described in WO 99/05914
TETA = triethylenetetramine available from Aldrich

What is claimed is:

1. A pesticidally active composition comprising:
1) at least one pesticidally active ingredient having at least one functionality which is reactive with singlet oxygen, and
2) at least one compound which acts as an activity enhancer which is distinct from the pesticidally active ingredient;
characterized in that the pesticidally active composition provides enhanced pesticidal protection and control by providing increased half life of the pesticidally active ingredient when compared to compositions without the activity enhancer.

2. The composition of claim 1 wherein the pesticidally active ingredient is selected from the group consisting of fungicides, insecticides, nematocides, miticides, biocides, termiticides, rodenticides, molluscides, arthropodicides, herbicides, attractants and pheromones.

3. The composition of claim 1 wherein the pesticidally active ingredient is a natural product selected from the group consisting of actinomycete bacteria, avermectins and derivatives thereof, spinosyns and derivatives thereof, sabadilla, veratrine, pyrethrum, pyrethrins, neem oil, azadirachtin, rotenone, ryania, ryanodine, Bacillus thuringiensis (B.t.), Bacillus subtilis, pheromones, and natural attractants.

4. The composition of claim 3, wherein the pesticidally active ingredient is selected form the group consisting of avermectins and derivatives thereof.

5. The composition of claim 3 wherein the pesticidally active ingredient is selected from spinosyns and derivatives thereof.

6. The composition of claim 3 wherein the pesticidally active ingredient is Bacillus thuringiensis (B.t.).

7. The composition of claim 3 wherein the pesticidally active ingredient is pyrethrum.

8. The composition of claim 1 wherein the activity enhancer is selected from the group consisting of carotenoids, tocopherols and chromanols, singlet oxygen deactivating proteins and amine containing compounds.

9. The composition of claim 8 wherein the activity enhancer is a carotenoid.

10. The composition of claim 8 wherein the activity enhancer is beta-carotene.

11. The composition of claim 8 wherein the activity enhancer is astaxanthin.

12. The composition of claim 8 wherein the activity enhancer is selected from tocopherols and chromanols.

13. The composition of claim 8 wherein the activity enhancer is alpha-tocopherol.

14. The composition of claim 8 wherein the activity enhancer is 6-hydroxy-2,5,7,8-tetramethylchroman-2-carboxylic acid.

15. The composition of claim 8 wherein the activity enhancer is a singlet oxygen deactivating protein.

16. The composition of claim 15 wherein the activity enhancer is egg albumin.

17. The composition of claim 15 wherein the activity enhancer is bovine serum albumin.

18. The composition of claim 8 wherein the activity enhancer is an amine containing compound.

19. The composition of claim 8 wherein the activity enhancer is selected from the group consisting of diazabicyclooctane, hexamethylenetetramine, tetraethylenepentamine, pentaethylenehexamine and triethylenetetramine.

20. The composition of claim 19 wherein the activity enhancer is pentaethylenehexamine.

21. The composition of claim 19 wherein the activity enhancer is tetraethylenepentamine.

22. The composition of claim 1 wherein the activity enhancer and the pesticidally active ingredient are present in the pesticidally active composition at a ratio of 100:1 or less.

23. A method of controlling, preventing or eliminating unwanted living organisms comprising applying the composition of claim 1 to the loci of said organisms.

24. The method of claim 23 wherein the unwanted living organism is a fungus.

25. The method of claim 23 wherein the unwanted living organism is an insect.

26. The method of claim 23 wherein the unwanted living organism is a weed.

27. The method of claim 23 wherein the unwanted living organism is a rodent.

28. The method of claim 23 wherein the unwanted living organism is a bacteria or other microbe.
